(19) United States
(12) Patent Application Publication
(10) Pub. No.: US 2015/0216490 A1
(43) Pub. Date: Aug. 6, 2015

(54) Radiolucent Transmitters for Magnetic Position Measurement Systems

(71) Applicant: Ascension Technology Corporation, Burlington, VT (US)

(72) Inventor: Wesley S. Ashe, Hinesburg, VT (US)

(21) Appl. No.: 14/608,594

(22) Filed: Jan. 29, 2015

Related U.S. Application Data
(60) Provisional application No. 61/934,394, filed on Jan. 31, 2014.

(51) Int. Cl. A61B 6/12; A61B 5/06

(52) U.S. Cl. CPC A61B 6/12; A61B 5/062

(57) ABSTRACT

Among other things, a device for use in a magnetic position measurement system includes a transmitter. The transmitter includes field generating elements having a low X-ray cross section. The field generating elements include at least one conductive spiral arranged in a sheet or a plate. The at least one spiral is planar. The transmitter also includes non-field generating regions surrounding the field generating elements.

### CLAIM OF PRIORITY

This application claims priority under 35 USC §119(e) to U.S. Patent Application Ser. No. 61/934,394, filed on Jan. 31, 2014, the entire contents of which are hereby incorporated by reference.

### TECHNICAL FIELD

This disclosure relates to radiolucent transmitters for magnetic position measurement systems.

### BACKGROUND

Magnetic position measurement systems are used to aid location of instruments and anatomy in medical procedures involving X-ray imaging devices. These systems utilize a magnetic transmitter in proximity to a magnetic sensor and the sensor can be spatially located relative to the magnetic transmitter. Magnetic transmitters employ construction methods which can result in uneven X-ray absorption across the transmitter, which can produce objectionable visual artifacts when the transmitter is placed in the X-ray imaging area. Also, the X-ray absorption through the magnetic field generating elements is orders of magnitude higher than that of the patient anatomy, and therefore, imaging the patient is typically not performed through the field generating elements. Additionally, parts of the X-ray imaging system, such as the image intensifier on a fluoroscope, can distort the magnetic fields and cause the magnetic positioning system to report incorrect sensor positions. As a result, magnetic transmitters are located carefully, and often the image intensifier is moved to obtain reliable position data. This leads to undesirable interruptions to the medical procedure, and imaging performance at a location which gives the best X-ray image quality can be compromised by the magnetic position measurement system.

### SUMMARY

The present disclosure describes a magnetic transmitter for use in a magnetic positioning system employed in conjunction with an X-ray imaging device. The transmitter, which includes magnetic field generating elements, is constructed such that, in some implementations, when X-ray passes through the transmitter, the transmitter and the magnetic field generating elements absorb up to approximately 10 percent of the X-ray. Furthermore, the X-ray absorption is made substantially uniform across the transmitter surface, including the field generating elements, allowing the transmitter to be placed in the x-ray imaging path without degrading image quality. The magnetic transmitter may be incorporated in an assembly that includes a low absorption, uniform X-ray attenuation magnetic shielding element. The shielding element can also be located in the X-ray imaging path, to reduce the distortion effect of nearby equipment, such as an image intensifier.

The magnetic transmitter may employ one or more types of geometries, such as being planar e.g., flat in nature. The magnetic transmitter may also be produced, adjusted, etc. to conform to different shapes, for example to be form-fitting relative to a patient's chest, back, abdomen, leg, head, etc. The magnetic transmitter can be achieved with a variety of designs, components, etc. such as a planar aluminum coil with a shield. As the planar aluminum coils and shield are malleable, they can be pressed or rolled into one or more desired shapes.

In order to detect position from one or more sensors in a measurement volume, a processing system may be used. The processing system can utilize one or more mathematical models of the magnetic field from the coils of the transmitter, or can use a pre-acquired map obtained using a magnetic sensor and a robotic positioning fixture, etc.

In one aspect, a device for use in a magnetic position measurement system includes a transmitter. The transmitter includes field generating elements. The field generating elements have a low X-ray cross section. The field generating elements include at least one conductive spiral arranged in a sheet or a plate. The at least one spiral is planar. The transmitter also includes non-field generating regions surrounding the field generating elements.

In another aspect, a method includes forming a transmitter. The transmitter includes field generating elements that have a low X-ray cross section. Forming the transmitter includes forming at least one planar conductive spiral in a sheet or plate. Forming the transmitter also includes surrounding the at least one planar conductive spiral with non-field generating regions.

In another aspect, a magnetic positioning system for use with an X-ray imaging system includes transducer elements constructed using one or more of aluminum, carbon, beryllium, or other conductive material that has a molecular weight of 27 or lower.

Implementations of the systems, devices, and methods may include one or more of the following features.

In some implementations, the field generating elements have an X-ray attenuation of 50% or less when the device is used with an X-ray imaging system.

In some implementations, the X-ray attenuation is uniform across an entire surface of the transmitter, including the field generating elements and the non-field generating regions.

In some implementations, the device also includes a support structure supporting the field generating elements. The support structure has a low X-ray cross section and includes carbon fibers, boron fibers, glass fibers, or other materials that have a molecular weight of 27 or lower.

In some implementations, the device also includes a conductive eddy current shield element. The shield element includes a metal having a molecular weight of 27 or lower and is located at least partly in an X-ray path. The shield element has an X-ray attenuation of 50% or less when the device is used with an X-ray imaging system.

In some implementations, spaces within each spiral are filled with a material having similar X-ray absorption to a material of the spiral.

In some implementations, the material includes aluminum having the same thickness as the spiral and laminated to a same base substrate as the spiral, or a different material whose thickness is adjusted such that the X-ray absorption matches that of the spiral.

In some implementation, the material includes a polymer matrix mixed with a material including powdered titanium dioxide. A proportion of the material is adjusted such that the X-ray attenuation matches that of the spiral.

In some implementations, the material in the spaces of the spiral includes a negative, mirror image spiral disposed adjacent to the spiral, such that the combined X-ray attenuation of the material through a surface of the transmitter is uniform.

In some implementations, the mirror image spiral is supported by a conductive or non-conductive substrate.

In some implementations, the field generating elements include a planar spiral coil formed of aluminum conductors and an attenuating material adjacent the conductors in spaces among the conductors. The attenuating material has a similar X-ray absorption to the aluminum conductors.

In some implementations, the spiral is an aluminum spiral coil on a polymer substrate.

In some implementations, the transmitter is configured to attach to an X-ray imaging equipment during an X-ray imaging procedure.

In some implementations, the transmitter is configured to flexibly deform into a desired shape.

In some implementations, the transmitter is operated as a receiver to sense magnetic fields generated by a magnetic transmitter.

In some implementations, the device also includes a sensor and a processor. The transmitter is operated to magnetically couple to the sensor. The sensor employs wireless re-transmission of magnetic fields generated by the transmitter. The transmitter is connected to the processor. The processor is capable of detecting characteristics of the re-transmission, an output position, and an orientation of the sensor.

In some implementations, the spirals are formed of square aluminum wires.

In some implementations, the method also includes positioning the transmitter such that an X-ray imaging region of interest is within a uniform attenuation boundary of the transmitter as an X-ray imaging equipment is operated and positioned.

In some implementations, forming the at least one spiral comprises photochemically etching a spiral pattern onto an aluminum polyester laminate sheet.

In some implementations, forming the transmitter comprises filling a material in spaces in each spiral in the form of a mirror image spiral.

In some implementations, the mirror image spiral is created as part of a shielding element, mechanical support, or an additional field generating spiral layer.

In some implementations, the transducer elements are coils.

In some implementations, the magnetic positioning system also includes a transmitter assembly. The transmitter assembly includes a support structure, a magnetic transmitter, and a shield. The magnetic transmitter includes the transducer elements.

In some implementations, the magnetic positioning system also includes a sensor and a processor.

In some implementations, the processor is configured to magnetically couple to the sensor, which employs wireless re-transmission of magnetic fields generated by the transmitter assembly. The processor is also configured to receive information from the transmitter assembly. The processor is also configured to detect characteristics of the re-transmission, an output position, and an orientation of the sensor.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic top view of an example magnetic transmitter.

FIG. 2A is a schematic cross-sectional side view of an example coil of a magnetic transmitter.

FIG. 2B is a schematic top view of an example coil of a magnetic transmitter.

FIG. 3 is a schematic diagram of a transmitter assembly.

FIG. 4 is a schematic diagram of an imaging system that includes a magnetic tracking system.

FIG. 5 is a flow chart depicting an example of a process for forming a transmitter.

### DETAILED DESCRIPTION

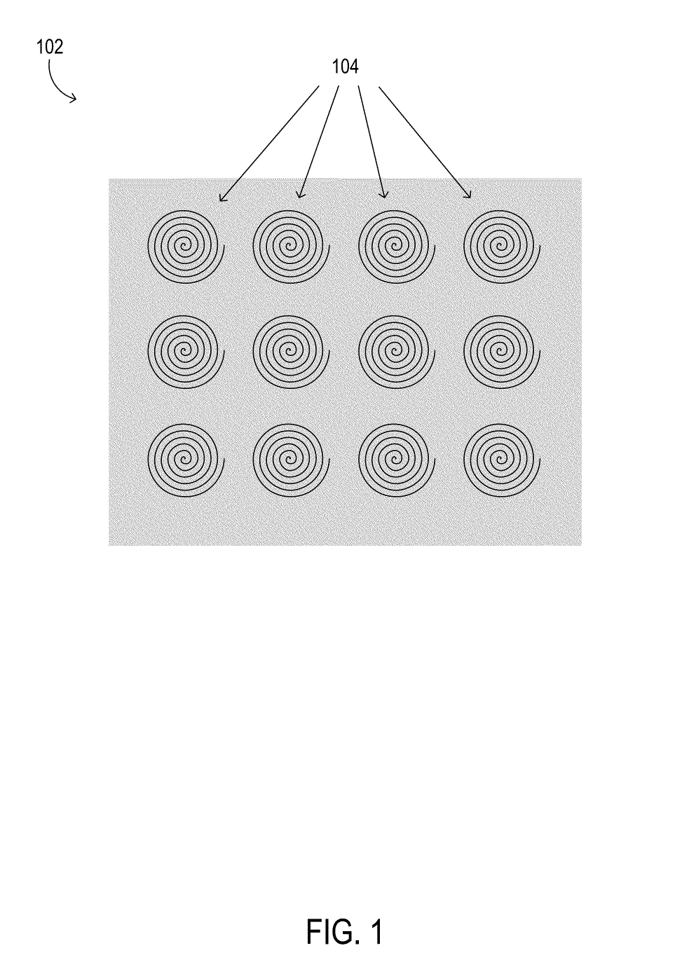

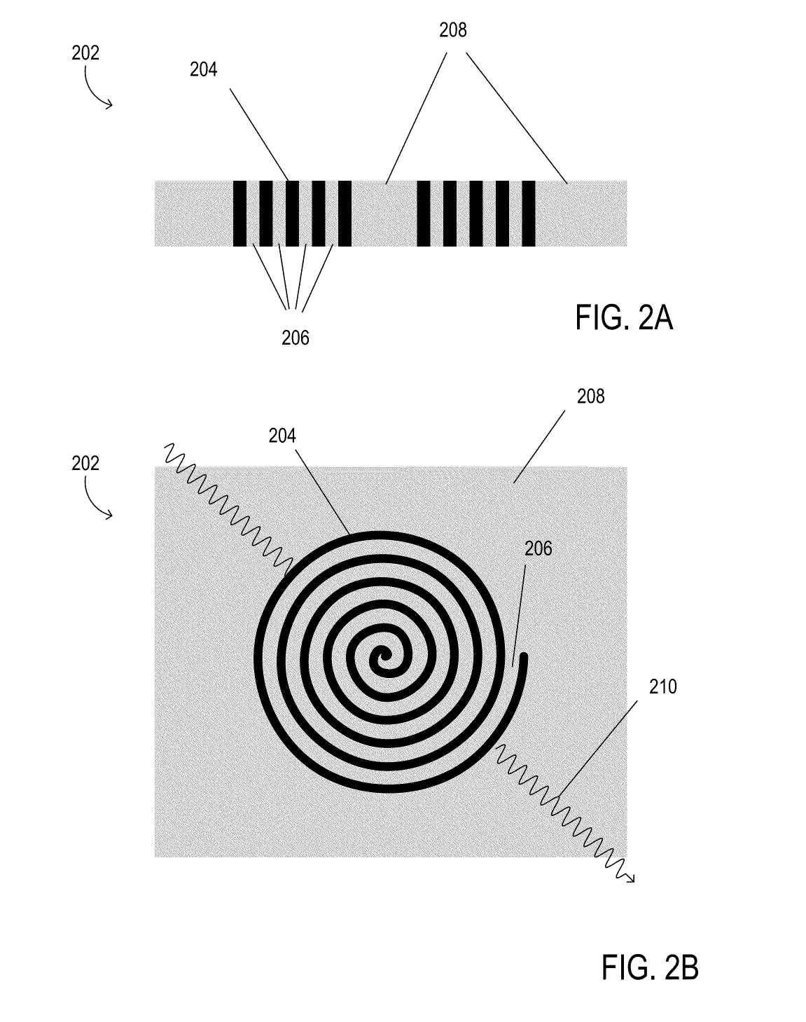

Referring to FIG. 1, a magnetic transmitter 102 includes one or more field generating elements 104. In the example shown in FIG. 1, the field generating elements 104 are in the form of planar spiral coils. In this particular example, the magnetic transmitter 102 includes twelve field generating elements 104 that employ a generally spiral geometry, although more or fewer field generating elements 104 may be used and distributed across the magnetic transmitter 102 in one or more patterns, designs, etc. The total number of field generating elements 104 can be chosen to provide desired tracking performance. In some examples, each field generating element 104 is about 0.5 mm thick and has an outer diameter of approximately 80 mm, although other dimensions are possible. A single conductor can be used to produce the spiral geometry, and the conductor can include a number of turns (e.g., 75 turns). To provide space between the turns, one or more separations may be implemented. For example, the field generating elements 104 can spaced apart by a particular distance (e.g., 10 mm), and can be arranged in a grid configuration (e.g., a 3×4 grid configuration), although other configurations are possible. The field generating elements 104 can be made of any suitable material. In some examples, the field generating elements 104 are made of aluminum conductors. Each field generating element 104 can have one or more layers (e.g., one layer, two layers, etc.). In cases in which the field generating elements 104 are coils, coil terminals at an outer edge of each of the layers have a spiral shape. In some implementations, the coil terminals are positioned adjacent to an edge of the magnetic transmitter 102 (e.g., such that the coil terminals are positioned away from the path of the X-ray 210 (shown in FIG. 2). The field generating elements 104 can have other dimensions chosen based on the desired properties of the magnetic transmitter 102.

FIGS. 2A and 2B show schematic side and top views, respectively, of an example of a coil 202. The coil 202 is one example of a field generating element 104 of FIG. 1. In some implementations, the coil 202 can be constructed by chemically etching a low molecular weight conductive substrate, (e.g., such as aluminum, carbon, beryllium, or other materials). In some examples, the conductive substrate has a molecular weight of no greater than 27, although other molecular weights are possible. A coil made of a low molecular weight material can reduce X-ray absorption when an X-ray 210 passes through the coil. A conductor 204 of the coil 202 can have multiple turns and can be formed by selectively removing material to form a spiral conductor pattern. The spiral may be circular, rectangular, or can have another shape chosen for desired magnetic field generating characteristics.

Spaces 206 between turns of the conductor 204 can be filled with a filler material. The filler material can have a similar X-ray absorption rate to the conductor 204. In some implementations, the conductor 204 has a higher X-ray absorption per unit distance along the X-ray 210 incident direction than the filler material, and a material having a relatively high molecular weight may be incorporated into the filler material such that the molecular weight of the filler material and the molecular weight of the conductor 204 are equal or approximately equal to each other. For example, if the filler material includes a polymer matrix, a weight percentage of the high molecular weight material can be chosen based on the formula:

%filler=(Aconductor-Apolymer)/(AhighMW-Apolymer),

where: %filler represents the weight percentage of the high molecular weight material; Aconductor represents X-ray attenuation per unit distance of the conductor 204; Apolymer represents X-ray attenuation per unit distance of the polymer matrix; and AhighMW represents X-ray attenuation per unit distance of the high molecular weight material. The high molecular weight material can be uniformly distributed throughout the polymer matrix. In situations in which the conductor 204 is aluminum, suitable high molecular weight materials can include titanium dioxide and/or zirconium oxide powders. An example of suitable filler materials includes a mix of Epoxy and titanium dioxide powder (e.g., about 89 wt % of Epoxy and about 11 wt % of titanium dioxide powder). The filler material can also include aluminum powder or other materials. In some implementations, the filler material that can reside in the spaces 206 has the same thickness as the conductor 204. In some implementations, the filler material that can reside in the spaces 206 has a different thickness as the conductor 204. The thickness of the filler material can be chosen such that the X-ray absorption of the filler material matches the X-ray absorption of the conductor 204 (e.g., based on the components of the filler material and the conductor 204). The conductor 204 and the filler material can provide relatively uniform attenuation of incident X-rays, thereby reducing visual artifacts in X-ray imagery. In some implementations, the filler material and the coil 202 formed of the conductor 204 are laminated to a same base substrate.

In some implementations, the filler material is applied to each layer of the coil 202 using a window squeegee. The filler material can then be given time to cure. The coil 202, including the conductor 204 and the filler material, forms a relatively flat surface. In some implementations, the coil 202 can be formed using one or more square insulated aluminum magnet wires as the conductor 204. Compared to round wires, in some implementations, the square wires can absorb incident X-ray more uniformly due to its uniform thickness. In some implementations, the insulation of the magnet wire is relatively thin, such that the spaces 206 are not resolved by the X-ray imaging device.

In some implementations, the spaces 206 between turns of the conductor 204 can be made small enough to be below the resolution of the X-ray imaging device. As a result, the X-ray 210 passing the spaces 206 and the conductor 204 of the coil 202 is attenuated uniformly. In such cases, the filler material in the spaces 206 may be optional. The spaces can be formed by laser machining a conductor substrate. Other high resolution material removal processes can also be implemented.

Non-field generating regions 208 of the magnetic transmitter 102 (shown in FIG. 1) can also be fabricated to have a similar X-ray absorption rate as the conductor 204 of the coil 202 and the filler material. Alternatively, the non-field generating regions 208 can be formed of the same material and can have a thickness that is similar to the thickness of the conductor 204 of the coil 202. The non-field generating regions 208 may be electrically connected to one or more of the coils 202 for supplying current pathways. If the excitation current provided to the conductor 204 is AC current, eddy currents may be formed in the non-field generating regions 208 (e.g., if the non-field generating regions 208 are conductive). To avoid the formation of eddy currents, the non-field generating regions 208 may be broken into smaller conductive islands and the spaces between the islands can be filled with a filler material (e.g., the same filler material used in the spaces 206). Using this construction technique, the X-ray 210 can be attenuated uniformly across the surface of the magnetic transmitter 102, leading to minimal shadowing of the resultant X-ray image. As a result, the ability of clinicians to interpret the X-ray image and identify patient anatomy using the magnetic transmitter 102 can be preserved.

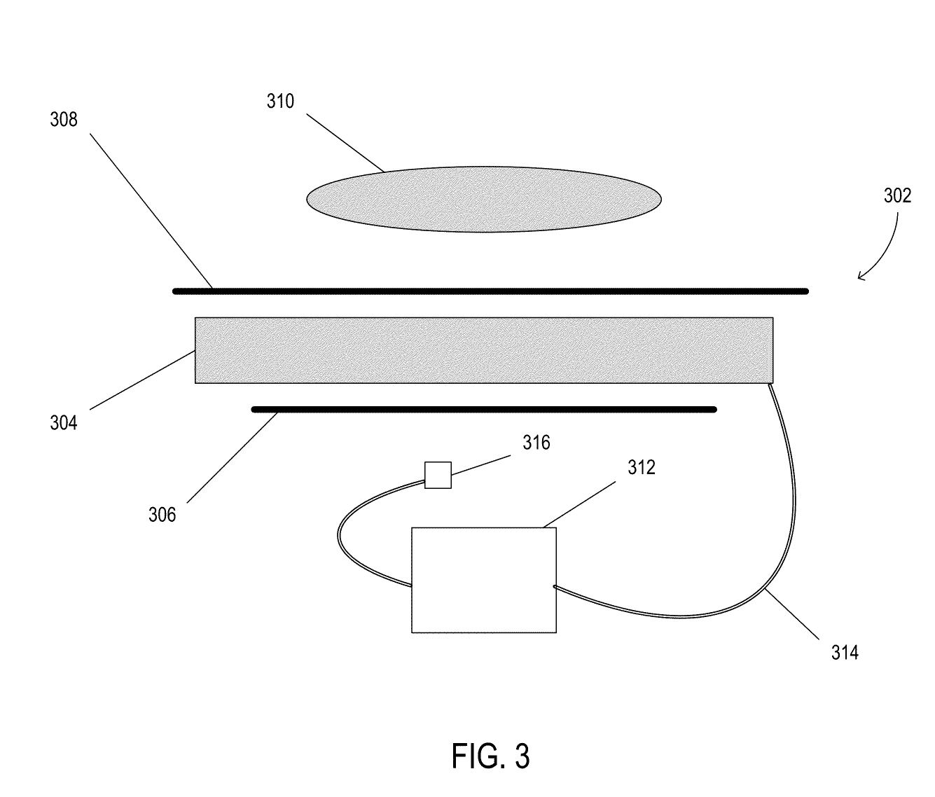

Referring to FIG. 3, a transmitter assembly 302 includes a support structure 304, a magnetic transmitter 306 positioned on one side of the support structure, and a shield 308 on an opposite side of the support structure 304. In use, the transmitter assembly 302 is positioned such that the shield 308 is positioned between the magnetic transmitter 306 and a distorting object 310. In some implementations, the distorting object 310 is a fluoroscopic image intensifier. The transmitter assembly 302 can be connected to a processor 312 by a cable 314. The processor 312 can receive information form the transmitter assembly 302 and determine the location of a sensor 316. In some implementations, the magnetic transmitter 306 is the same magnetic transmitter 102 shown in FIG. 1.

The magnetic transmitter 306 can be attached to the support structure 304. The magnetic transmitter 306 can include one or more field generating elements (e.g., the field generating elements 104 of FIG. 1). In some implementations, the field generating elements are coils (e.g., the coil 202 of FIGS. 2A and 2B). In some implementations, each coil has multiple layers. In some implementations, the total thickness of the magnetic transmitter 306 is 0.5 mm, with each coil being formed using two 0.25 mm thick aluminum layers.

In some examples, the support structure 304 can be 5 mm thick and can be made from carbon fibers or glass fibers. The carbon fibers may have a molecular weight of 12 and may be able to provide a relatively lower X-ray attenuation and higher strength as compared to the glass fibers. The carbon fiber can also provide good thermal conductivity, which can help move heat generated by the magnetic transmitter 306 away from the patient. The support structure 304 can provide the transmitter assembly 302 with strength. However, in some implementations, the support structure 304 may be omitted if additional strength is not necessary. The omission of the support structure 304 may be useful if the magnetic transmitter 306 needs to conform to an anatomical shape. If a more flexible magnetic transmitter 306 is desired, a relatively soft polymer (e.g., polyurethane) may be used to form the support structure 304.

In implementations in which the support structure 304 is used, the support structure provides structural support for the transmitter assembly 302. The support structure 304 can be made of any material suitable for providing sufficient support without interfering with the functions of the magnetic transmitter 306. In some implementations, the support structure 304 includes one or more substructures and/or one or more layers. In some implementations, the support structure has a relatively low X-ray cross section and includes one or more of carbon fibers, boron fibers, glass fibers, and other materials that have a molecular weight of 27 or lower.

In some examples, the shield 308 can be an eddy current shield. In some implementations, the shield 308 is made from a 0.5 mm thick aluminum layer and is attached to the support structure 304. The shield 308 can reduce magnetic field coupling to adjacent conductive or ferromagnetic distorting objects (e.g., such as distorting object 310) and thus can improve the accuracy of the processor 312 in determining the position of the sensor 316. The thickness of the shield 308 can be chosen such that the X-ray absorption and the skin depth at the excitation frequency of the magnetic transmitter 306 is appropriate. For example, the thickness of the shield 308 can be chosen such that the shield 308 does not overly absorb an X-ray passing through the transmitter assembly 302. In some implementations, the excitation frequency is 3,200 Hz and the shield 308 is 0.5 mm thick.

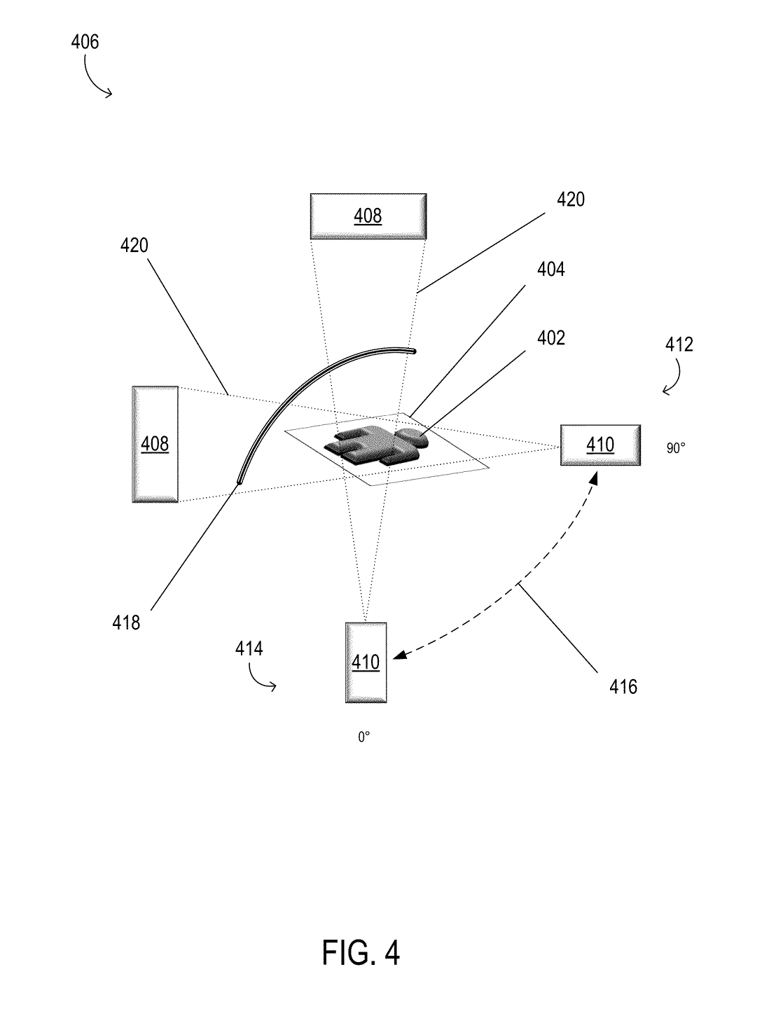

Referring to FIG. 4, in X-ray imaging, a patient 402 is positioned on a surface 404. An imaging system 406 including an X-ray image intensifier 408 and an X-ray source 410 are arranged on opposite sides of the patient 402. In some examples, the X-ray image intensifier 408 and the X-ray source 410 form a “C arm” for a fluoroscope. The X-ray image intensifier 408 and the X-ray source 410 can rotate together between a 90 degree position 412 and a 0 degree position 414 along a motion arc 416. A magnetic tracking system including a transmitter assembly 418, a sensor, and a processor can be used in connection with the imaging system 406. The transmitter assembly 418, the sensor, and the processor may have properties similar to the transmitter assembly 302, the sensor 316, and the processor 312 described above with reference to FIG. 3. For example, the transmitter assembly 418 of FIG. 4 includes the support structure 304, the magnetic transmitter 306, and the shield 308 shown in FIG. 3.

Referring to FIGS. 3 and 4, the transmitter assembly 418 may be formed into a shape that fits anatomical contours of the patient 402. For example, the transmitter assembly 418 may have a curved shape. The transmitter assembly 418 may further be constructed such that it positioned adjacent to an X-ray imaging area of interest as the X-ray image intensifier 408 and the X-ray source 410 rotate to desired viewing positions during imaging of the patient 402. X-rays emitted from the X-ray source 410 do not extend beyond the transmitter assembly 418 when the X-ray source 410 is in either the 0 degree position 414 or the 90 degree position 412, as signified by a cone 420 that represents a projection range of the X-ray source 410. Such an assembly can prevent a shadow from appearing in the X-ray image. The positioning of the various components of the imaging system 406 and the transmitter assembly 418 can also allow the shield 308 of the transmitter assembly 418 to remain between the magnetic transmitter 306 and the X-ray image intensifier 408, thereby reducing magnetic field distortions caused by the X-ray image intensifier 408.

An automatic gain control in the C arm can increase the X-ray intensity automatically to compensate for the X-ray attenuation cause by the transmitter assembly 418. In some implementations, the attenuation is approximately 10 percent. The increase in X-ray intensity can result in a raised X-ray dose rate to the patient 402 when the X-ray imaging is performed. However, in some implementations, the position information received from the processor 312 can allow navigation of the patient 402 using only the magnetic tracking system during a significant portion of the imaging procedure. In some implementations, the X-ray source 412 can be turned off. It is thus possible to greatly reduce the overall radiation dose experienced by the patient 402 and surrounding personnel (e.g., operators, nurses, doctors, and other medical professionals).

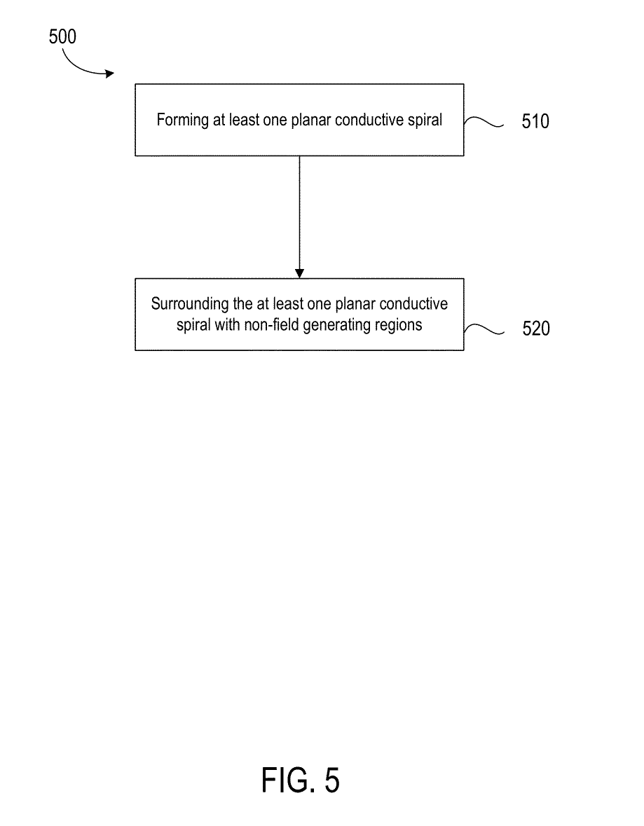

FIG. 5 is a flow chart depicting an example of a process for forming a transmitter (e.g., the magnetic transmitter 102 of FIG. 1 or the magnetic transmitter 306 of FIG. 3). The transmitter can include one or more field generating elements. In some implementations, the field generating elements have a relatively low X-ray cross section. At step 510, at least one planar conductive spiral is formed (e.g., the coil 202 shown in FIG. 2). In some implementations, the at least one planar conductive spiral is formed in a sheet or a plate. At step 520, the at least one planar conductive spiral is surrounded with non-field generating regions (e.g., the non-field generating regions 208 shown in FIG. 2).

The imaging system described above can be implemented using software for execution on a computer. For instance, the software forms procedures in one or more computer programs that execute on one or more programmed or programmable computer systems (which may be of various architectures) each including at least one processor, at least one data storage system (including volatile and non-volatile memory and/or storage elements), at least one input device or port, and at least one output device or port.

The software may be provided on a storage medium, such as a CD-ROM, readable by a general or special purpose programmable computer or delivered (encoded in a propagated signal) over a communication medium of a network to the computer where it is executed. All of the functions may be performed on a special purpose computer, or using special-purpose hardware, such as coprocessors. The software may be implemented in a distributed manner in which different parts of the computation specified by the software are performed by different computers. Each such computer program is preferably stored on or downloaded to a storage media or device (e.g., solid state memory or media, or magnetic or optical media) readable by a general or special purpose programmable computer, for configuring and operating the computer when the storage media or device is read by the computer system to perform the procedures described herein. The inventive system may also be considered to be implemented as a computer-readable storage medium, configured with a computer program, where the storage medium so configured causes a computer system to operate in a specific and predefined manner to perform the functions described herein.

A number of embodiments have been described. Nevertheless, it will be understood that various modifications may be made without departing from the spirit and scope of the invention. For example, it is possible to mount the transmitter assembly directly upon the X-ray image intensifier or the X-ray source 16. In some implementations, the transmitter assembly can be used inside a CT scanner bore or a diagnostic chest X-ray using similar construction techniques to those described above. In some implementations, the transmitter assembly can be fastened to the patient using adhesive strips before imaging starts. In some implementations, padding such as foam can be used to customize the fit of the transmitter assembly to the patient so that a standard transmitter size can securely fit multiple anatomies. In some implementations, small holes can be provided in the transmitter assembly to allow for access to the patient (e.g., for introducing catheters or other medical devices through the transmitter assembly).

Other embodiments are within the scope of the following claims.

What is claimed is:

1. A device for use in a magnetic position measurement system, the device comprising:
a transmitter comprising
field generating elements having a low X-ray cross section, wherein the field generating elements include at least one conductive spiral arranged in a sheet or a plate, and wherein the at least one spiral is planar, and
non-field generating regions surrounding the field generating elements.

2. The device of claim 1, wherein the field generating elements have an X-ray attenuation of 50% or less when the device is used with an X-ray imaging system.

3. The device of claim 1, wherein the X-ray attenuation is uniform across an entire surface of the transmitter, including the field generating elements and the non-field generating regions.

4. The device of claim 1, comprising a support structure supporting the field generating elements, wherein the support structure has a low X-ray cross section and includes carbon fibers, boron fibers, glass fibers, or other materials that have a molecular weight of 27 or lower.

5. The device of claim 1, comprising a conductive eddy current shield element, wherein the shield element comprises a metal having a molecular weight of 27 or lower and is located at least partly in an X-ray path, and wherein the shield element has an X-ray attenuation of 50% or less when the device is used with an X-ray imaging system.

6. The device of claim 1, wherein spaces within each spiral are filled with a material having similar X-ray absorption to a material of the spiral.

7. The device of claim 6, wherein the material comprises aluminum having the same thickness as the spiral and laminated to a same base substrate as the spiral, or a different material whose thickness is adjusted such that the X-ray absorption matches that of the spiral.

8. The device of claim 6, wherein the material comprises a polymer matrix mixed with a material including powdered titanium dioxide, with a proportion of the material adjusted such that the X-ray attenuation matches that of the spiral.

9. The device of claim 6, wherein the material in the spaces of the spiral comprises a negative, mirror image spiral disposed adjacent to the spiral, such that the combined X-ray attenuation of the material through a surface of the transmitter is uniform.

10. The device of claim 9, wherein the mirror image spiral is supported by a conductive or non-conductive substrate.

11. The device of claim 1, wherein the field generating elements comprise a planar spiral coil formed of aluminum conductors and an attenuating material adjacent the conductors in spaces among the conductors, the attenuating material having a similar X-ray absorption to the aluminum conductors.

12. The device of claim 1, wherein the spiral is an aluminum spiral coil on a polymer substrate.

13. The device of claim 1, wherein the transmitter is configured to attach to an X-ray imaging equipment during an X-ray imaging procedure.

14. The device of claim 1, wherein the transmitter is configured to flexibly deform into a desired shape.

15. The device of claim 1, wherein the transmitter is operated as a receiver to sense magnetic fields generated by a magnetic transmitter.

16. The device of claim 1, comprising a sensor and a processor, wherein the transmitter is operated to magnetically couple to the sensor, which employs wireless re-transmission of magnetic fields generated by the transmitter, the transmitter is connected to the processor, which is capable of detecting characteristics of the re-transmission, an output position, and an orientation of the sensor.

17. The device of claim 1, wherein the spirals are formed of square aluminum wires.

18. A method comprising:
forming a transmitter comprising field generating elements having a low X-ray cross section comprising
forming at least one planar conductive spiral in a sheet or plate, and
surrounding the at least one planar conductive spiral with non-field generating regions.

19. The method of claim 18, comprising positioning the transmitter such that an X-ray imaging region of interest is within a uniform attenuation boundary of the transmitter as an X-ray imaging equipment is operated and positioned.

20. The method of claim 18, wherein forming the at least one spiral comprises photochemically etching a spiral pattern onto an aluminum polyester laminate sheet.

21. The method of claim 18, wherein forming the transmitter comprises filling a material in spaces in each spiral in the form of a mirror image spiral.

22. The method of claim 21, wherein the mirror image spiral is created as part of a shielding element, mechanical support, or an additional field generating spiral layer.

23. A magnetic positioning system for use with an X-ray imaging system, the magnetic positioning system comprising:
transducer elements constructed using one or more of aluminum, carbon, beryllium, or other conductive material that has a molecular weight of 27 or lower.

24. The magnetic positioning system of claim 23, wherein the transducer elements are coils.

25. The magnetic positioning system of claim 23, further comprising a transmitter assembly that includes a support structure, a magnetic transmitter, and a shield, wherein the magnetic transmitter includes the transducer elements.

26. The magnetic positioning system of claim 25, further comprising a sensor and a processor.

27. The magnetic positioning system of claim 26, wherein the processor is configured to:
magnetically couple to the sensor, which employs wireless re-transmission of magnetic fields generated by the transmitter assembly;
receive information from the transmitter assembly; and
detect characteristics of the re-transmission, an output position, and an orientation of the sensor.
